(19) United States
(12) Patent Application Publication
(10) Pub. No.: US 2012/0010242 A1
(43) Pub. Date: Jan. 12, 2012

(54) LOW DOSE PIPAMPERONE IN TREATING MOOD DISORDERS

(75) Inventor: Erik Buntinx, Alken (BE)

(73) Assignee: ALKERSTRAAT 30 A, ALKEN (BE)

(21) Appl. No.: 13/258,020

(22) PCT Filed: Mar. 30, 2010
(86) PCT No.: PCT/EP10/54249

§ 371 Date: Sep. 21, 2011

(30) Foreign Application Priority Data
Mar. 20, 2009 (EP) 09156750.3

(51) Int. Cl. A61K 31/4545; A61P 25/00; C07D 401/04

(52) U.S. Cl. 514/316; 546/189

(57) ABSTRACT

The present invention relates to the use of low dose pipamperone and compositions comprising the same for the treatment of mood disorders.

The invention relates to the field of psychiatry. More specifically, the invention relates to the use of pipamperone in treating mood disorders.

Conventionally, mental disorders are divided into types based on criteria sets with defining features.

DSM-IV (American Psychiatric Association, (1993—ISBN 0-89042-061-0)) is the in the art well-known gold standard of such a categorical classification. In DSM-1V, there is no assumption that each category of mental disorder is a completely discrete entity with absolute boundaries dividing it from other mental disorders or from no mental disorder. There is also no assumption that all individuals described as having the same mental disorder are alike in all important ways. Individuals sharing a diagnosis are likely to be heterogeneous even in regard to the defining features of the diagnosis. Thus, the categorical defined mental disorders such as mood disorders are having an external and even internal variable co-incidence of symptoms concerning mood.

In a dimensional system, clinical presentations are classified based on quantification of attributes, i.e. dysfunctions rather than the assignment to categories. This works best in describing phenomena, which are distributed continuously and which do not have clear boundaries. Emotion dysregulation is known as such an attribution or dysfunction that plays an important role in the development and course of mental disorders (Gross, J. J. & Munoz, R. F., 1995, Clinical Psychology: Science and Practice, 2, 151-164; Mennin, D. S., Heimberg, R. G., Turk, C. L. & Fresco, D. M., 2002, Clinical Psychology: Science and Practice, 9, 85-90; Linehan, M. M., 1993, New York, The Guilford Press; Gratz, K. L., Roemer, L., 2001 & 2004, Annual meeting of the Association for Advancement of Behavior Therapy, Nov. 2001 & Journal of Psychopathology and Behavioral Assessment, Vol. 26, No. 1, March 2004) besides behavioural and cognitive dysfunctions.

Finally, in the biological system, mental disorders are defined on other levels of abstraction than in the categorical and dimensional system. Structural pathology (e.g. amyloid plaques in Alzheimer Disease), etiology (e.g. HIV Dementia) and deviance from a physiological norm (e.g. reduced cerebral blood flow) are often used as indicative biological markers for a mental disorder. The underlying dysregulation of various neurotransmittor systems (glutaminergic, GABAergic, cholinergic, monoaminergic (nor-adrenergic, dopaminergic, serotonergic), etc.) is the in the art used model for the explanation of the biological determinants of the clinical presentation of mental disturbances.

Mood disorders include major depressive disorder, bipolar disorder (combining episodes of both mania and depression) and dysthymia. As a group, mood disorders are one of the most common mental illnesses in the general population. Approximately 8% of adults will experience major depression at some time in their lives, while approximately 1% will experience bipolar disorder. Other studies have reported that between 3% and 6% of adults will experience dysthymia during their lifetime, and that between 0.6% and 1% of adults will have a manic episode during their lifetime. Because of their high prevalence, economic cost, risk of suicide and loss of quality of life, mood disorders present a serious public health concern in society. Also, depression and mania cause significant distress and impairment in social, occupational, educational or other important areas of functioning. According to the World Health Organization (WHO), major depression is the fourth leading cause of disability adjusted life years (DALYs) in the world. Major depression is the leading cause of years of life lived with disability (YLD) and bipolar is the sixth leading cause. Social and economic effects of mood disorders include functional impairment, disability or lost work productivity, and increased use of health services.

Depression is a serious mood disorder which affects millions of people, while the number of people being diagnosed with depression has increased dramatically. There is no single known cause of depression. Rather, it likely results from a combination of genetic, biochemical, environmental, and psychological factors. Nevertheless, important neurotransmitters appear to be out of balance. In particular, serotonin signaling is affected in depressed patients. Hence, depression is treated with antidepressants which work to normalize neurotransmitters, notably serotonin and norepinephrine. Other antidepressants work on the neurotransmitter dopamine. Although it is found that these particular neurotransmitters are involved in regulating mood, it is uncertain of the exact ways in which they work.

Hence, because of their high prevalence, apart from the impact on the individual itself and his relation, mood disorders have a major effect on economy. This effect is dual in nature first, with the associated loss of productivity in the workplace due to absenteeism and diminished effectiveness; and second, with the high health care costs attributable to primary care visits, hospitalizations and medication. At the individual and family level, the loss of income and cost of medication create a strain on the family financial resources. Hence, mood disorders have a major economic impact through associated health care costs as well as lost work productivity. Accordingly, there is a substantial need to diagnose and treat these disorders.

Mainstream treatment for mood disorders consists of the prescription of antidepressants. The newest and most popular types of antidepressant medications are called selective serotonin reuptake inhibitors (SSRIs). SSRIs include fluoxetine (Prozac), citalopram (Celexa), sertraline (Zoloft) and several others. Serotonin and norepinephrine reuptake inhibitors (SNRIs) are similar to SSRIs and include venlafaxine (Effexor) and duloxetine (Cymbalta). SNDRIs are so-called triple reuptake inhibitors, which elevate extracellular plasma concentrations of all three monoamine neurotransmitters, serotonin, dopamine and norepinephrine in the synaptic cleft. No SNDRIs are yet on the market, although the one of these agents, GlaxoSmithKline's NS2359 is currently in clinical trials, and other compounds such as brasofensine and tesofensine are under development.

Antidepressants are also known to cause side effects. The most common side effects associated with SSRIs and SNRIs include: headache, nausea, insomnia, nervousness, agitation and sexual problems. For instance, citalopram is a commonly used SSRI. Citalopram can have a number of adverse effects. In clinical trials, over 10% of patients reported one or more of the following side effects: fatigue, drowsiness, dry mouth, increased sweating (hyperhidrosis), trembling, headache, dizziness, sleep disturbances, insomnia, cardiac arrhythmia, hallucinations, blood pressure changes, nausea and/or vomiting, diarrhea, heightened anorgasmia in females, impotence and ejaculatory problems in males. In some cases, allergic reactions, convulsions, mood swings, anxiety and confusion have been reported. Also sedation may be present during treatment of antidepressants, for instance citalopram.

For all classes of antidepressants, patients must take regular doses for at least three to four weeks before they are likely to experience a full therapeutic effect. However, clinical or real effectiveness of psychopharma is very rare via common pooping-out; many treatment-refractory patients and up to half of patients fail to attain remission (S. M. Stahl, Essential Psychopharmacology, Depression and Bipolar Disorders, 151, University Press; 2 edition (Jun. 15, 2000); ISBN: 0521646154). Implications of not attaining remission for Mental Disorders are increased relapse rates, continuing functional impairment and increased suicide rate (S. M. Stahl, ibid). Clinical causes of not attaining remission by the Current Psychopharmacological Compounds are inadequate early treatment, underlying emotion dysregulation (affecting instability—hypersensitivity—hyperaesthesia—dissociative phenomena, etc.) and competitive antagonism.

Hence, there is a need for more efficient, selective and efficacious medicaments for treating mood disorders.

WO 2005/053796 relates to low doses of antagonists of 5-HT2A and D4 receptors in order to augment the effects of second compounds in mental disorders. One of these antagonists is pipamperone. WO 2005/053796 describes no effect of pipamperone independently of second compounds on treating mental disorders.

The instruction leaflet from the manufacturer Janssen Cilag B. V details psychoses and the symptomatic treatment of serious forms of agitation and anxiety as the therapeutic indications for pipamperone. The leaflet warns against the use of pipamperone in depression. The recommended initial dose is 40 to 80 mg pipamperone a day. If necessary the dose may be increased to a maximum of 360 mg pipamperone per day.

The present inventor surprisingly found that pipamperone at a dose of more than 0.1 to about 20 mg pipamperone per day is effective in treating mood disorders.

The present invention relates to the use of pipamperone or a pharmaceutically acceptable salt thereof for the preparation of a medicament for treating a mood disorder, wherein said pipamperone or a pharmaceutically acceptable salt is to be administered to a patient in a daily dose ranging between 0.1 and about 20 mg of the active ingredient.

The present invention relates also to a pharmaceutical composition comprising pipamperone for use in treating mood disorders, wherein said pipamperone is to be administered to a patient in a daily dose ranging between 0.1 and about 20 mg of the active ingredient.

The present invention relates also to a pharmaceutical composition comprising pipamperone, or a pharmaceutically acceptable salt thereof, at between 0.1 mg and about 20 mg of the active ingredient, preferably in that it is a unit dose preparation containing 0.1 to about 20 mg pipamperone, even more preferably, in that it is a unit dose preparation containing not more than 15 mg pipamperone, and even more preferably, in that it is a unit dose preparation containing not more than 12.5 mg pipamperone, preferably 10 mg. The present invention relates also to the pharmaceutical composition as described above, characterised in that it is an oral formulation, preferably a tablet.

The present invention relates also to tablets comprising between 0.1 and about 20 mg pipamperone or a pharmaceutically acceptable salt thereof, and optionally lactose, corn starch, saccharose, talc, and/or magnesium-stearate.

The present invention relates also to the use of pipamperone comprising between 0.1 and about 20 mg pipamperone for the preparation of a pharmaceutical composition, preferably in that pipamperone is used as pipamperone dihydrochloride or pipamperone acetate, even more preferably, in that the pharmaceutical composition is for treatment of a mood disorder, in particular Major depressive disorder, including Major depressive episode, Atypical depression, Melancholic depression, Psychotic depression, Depressive Disorder Not Otherwise Specified, Depression (mood), Postpartum depression, Dysthymia, Adjustment disorder with depressed mood, or Seasonal affective disorder (SAD), and even more preferably in that the pharmaceutical composition is for treatment of major depressive disorder.

The present invention relates also to the use as described above, characterised in that the pharmaceutical composition is for treatment of treatment refractory patients to an antidepressant, such as an SSRI, SNDRI or SNRI, preferably in that the pharmaceutical composition is for treatment of major depression in treatment refractory patients to an SSRI, SNDRI or SNRI.

The present invention relates also to the use of pipamperone for the treatment of a mood disorder, preferably major depressive disorder characterised in that it is used in a daily dose of less than 20 mg of pipamperone, and to the use of pipamperone for the treatment of a mood disorder, preferably major depressive disorder characterised in that it is used in a daily dose of 15 mg or less of pipamperone, and to the use of pipamperone for the treatment of a mood disorder, preferably major depressive disorder characterised in that it is used in a daily dose of 10 mg of pipamperone.

Finally, the present invention relates to a method for treating mood disorder comprising administering pipamperone between 0.1 and less than 20 mg per day.

Before the present method and products of the invention are described, it is to be understood that this invention is not limited to particular methods, components, products or combinations described, as such methods, components, products and combinations may, of course, vary. It is also to be understood that the terminology used herein is not intended to be limiting, since the scope of the present invention will be limited only by the appended claims.

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. It will be appreciated that the terms “comprising”, “comprises” and “comprised of” as used herein comprise the terms “consisting of”, “consists” and “consists of”.

The recitation of numerical ranges by endpoints includes all numbers and fractions subsumed within the respective ranges, as well as the recited endpoints.

The term “about” as used herein when referring to a measurable value such as a parameter, an amount, a temporal duration, and the like, is meant to encompass variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” refers is itself also specifically, and preferably, disclosed.

All documents cited in the present specification are hereby incorporated by reference in their entirety.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention.

Mood disorders, such as depression, are commonly treated with serotonin re-uptake inhibitors. Unfortunately, however, these compounds can give rise to side effects in use, including sedation. Moreover, a substantial problem in most treatment of mental disorders is the non-response to serotonin re-uptake inhibitors. Also the onset of the therapeutic effect can be delayed undesirable.

A problem to be solved by the present invention is thus the provision of a more efficient therapy and efficient, highly selective and efficacious medicaments for treating mental disorders, in particular mood disorders, such as depression.

The common mode of action of SRIs is to block or substantially decrease the rate by which neurotransmitters, i.e. serotonin, are reabsorbed into the presynaptic neuron, leaving a net gain in the concentration of neurotransmitter in the synapse. This increases the probability and frequency of neurotransmitter binding to postsynaptic neurotransmitter receptors. The common SRIs are directed against the serotonin transporter (SERT). SERT is an integral membrane protein that transports the neurotransmitter serotonin from synaptic spaces into presynaptic neurons. This transport of serotonin by the SERT protein terminates the action of serotonin and recycles it in a sodium-dependent manner. SERT belongs to NE, DA, SERT monoamine transporter family, and spans the plasma membrane 12 times.

Although not be bound by any theory, the present inventor hypothesized that mood disorders may not only be combated by blocking or inhibiting the serotonin transporter, and thus increasing serotonin availability in the synaptic cleft, but also by blocking or decreasing inhibitory feedback mechanisms. In particular, in response to a short term increase of neurotransmitter concentration in the synapses, the genes and neuronal receptors reshape, due to a web of mutually inhibitory feed-back mechanisms. In combination with assessing dissociation constants (K_{d}) and pKi modelling, the present inventor found that a low dose treatment with pipamperone having a high selective 5-HT_{2A} and D4 antagonist activity leads to an increased remission of the underlying emotional dysregulation. In particular, the present inventor surprisingly found that pipamperone at an unconventionally low dose of less than 20 mg/day is efficacious in treating patients suffering from mood disorders, especially patients which are refractory to serotonin re-uptake inhibitors (SRIs), such as SSRIs, SNDRIs and SNRIs.

The term “serotonin re-uptake inhibitor” or “SRI” as used herein refers to any compound elevating the extracellular plasma concentration of serotonin in the synaptic cleft by blocking or inhibiting the function of SERT. The term “SRI” does not exclude the effects of these compounds on any other compound, e.g. other neurotransmitters. In particular, the term “SRI” includes SSRIs, SNDRIs and SNRIs.

Mood disorders can be diagnosed using criteria found in the American Psychiatric Association's revised fourth edition of the Diagnostic and Statistical Manual of Mental Disorders (DSM-IV-TR), and the WHO's International Statistical Classification of Diseases and Related Health Problems (ICD-10). DSM-IV sets forth diagnostic criteria, descriptions and other information to guide the classification and diagnosis of mental disorders and is commonly used in the field of neuropsychiatry. It is for instance available on the internet under: http://www.behavenet.com/ capsules/disorders/ dsm4tr.htm.

The mood disorders grouped under the DSM-IV include major depressive disorder, dysthymic disorder, bipolar disorder, cyclothymic disorder, mood disorder due to a general medical condition and substance induced mood disorder. For each of these mood disorders there are specific criteria that a person's symptoms must meet in order to receive a diagnosis.

The “depressive condition”, “depression” or “recurrent depressive disorder”, which are used interchangeably herein. The term “depression” according to the invention includes Major depressive disorder, Major depressive episode, Atypical depression, Melancholic depression, Psychotic depression, Depressive Disorder Not Otherwise Specified, Depression (mood), Postpartum depression, Dysthymia, Adjustment disorder with depressed mood, Seasonal affective disorder (SAD), all as known in the art.

Depression can be scored by e.g. the Hamilton Depression Rating Scale (HDRS or HAMD) (Hamilton M. A rating scale for depression. J Neurol Neurosurg Psychiatry 196023:56-62) or the Montgomery-Asberg Depression Rating Scale (abbreviated MADRS). There is, however, a high degree of statistical correlation between scores on the two measures. The Clinical Global Impression (CGI) rating scales are commonly used measures of symptom severity, treatment response and the efficacy of treatments in treatment studies of patients with mental disorders. The CGI - Severity scale (CGI-S) is a 7-point scale which can be used by the clinician to rate the severity of the patient's illness at the time of assessment, relative to the clinician's past experience with patients who have the same diagnosis.

In particular, the present invention relates to the use of pipamperone comprising between 0.1 and less than 20 mg for the preparation of a pharmaceutical composition or medicament, characterised in that the pharmaceutical composition or medicament is for treatment of a mood disorder.

In particular, the present invention relates to the use of pipamperone comprising between 0.1 and less than 20 mg for the preparation of a pharmaceutical composition, characterised in that the pharmaceutical composition is for treatment of mood disorders, in particular, Major depressive disorder, including Major depressive episode, Atypical depression, Melancholic depression, Psychotic depression, Depressive Disorder Not Otherwise Specified, Depression (mood), Postpartum depression, Dysthymia, Adjustment disorder with depressed mood, or Seasonal affective disorder (SAD), in particular for treatment of major depressive disorder.

The high selective affinity of pipamperone towards the 5-HT2A receptor and the D4 receptor is reflected in the low dosage which is needed for the treatment of mood disorders.

Pipamperone has both a selective affinity for the 5-HT2A receptor with a pKi value equal to or higher than 8 towards the 5-HT2A receptor and less than 8 towards other 5HT receptors, and a selective affinity for the D4 receptor with a pKi value equal to or higher than 8 towards the D4 receptor and less than 8 towards other dopamine receptors (see Table 1). Pipamperone is the conventional name given for the compound of the formula 1′-[3-(p-Fluorobenzoyl)propyl]-[1,4′-bipiperidine]-4′-carboxamide. Pipamperone is also the active ingredient of for instance the commercially available Dipiperon (Janssen, Cilag B. V).

According to the leaflet of the manufacturer, the therapeutic indications for pipamperone are psychoses and the symptomatic treatment of serious forms of agitation and anxiety only. Pipamperone is contraindicated for depression. For adults it is recommended to start with 40 to 80 mg a day. If necessary the dose may be increased to a maximum of 360 mg per day. In conventional pipamperone treatment, the active ingredient is available in tablets of 40 mg per tablet or in solutions of 2 mg per drop. Conventional usage of high doses ranging from 40 to 360 mg is prescribed. For instance, for children up to the age of 14, doses corresponding with 2 to 6 mg per kg body weight are conventionally prescribed.

The amount of pipamperone required to produce the efficacious effect will, of course, vary and is ultimately at the discretion of the medical practitioner. The factors to be considered include the route of administration and nature of the formulation, the patient's body weight, age and general condition and the nature and severity of the disease to be treated. Preferred dosages which, according to the invention, have been shown to be effective for treating the mood disorder range between about 0.1 and about 20 mg pipamperone per day. Preferably, about 0.1, 0.25, 0.5, 1, 1.5, 2, 2.5, 3, 3.5, 4, 4.5, 4.8, 4.9, 5, 5.5, 6, 6.5, 7, 7.5, 8, 8.5, 9, 9.5, 10, 11, 12, 12.5, 13, 14, 15, 16, 17, 17.5, 18, 19, 19.5 and 20 mg pipamperone per day is used.

In a preferred embodiment, dosages of more than 0.1 mg to about 10 mg per day are used in the treatment of mood disorders, preferably in treating children and elderly patients, in particular, about 0.1, 0.25, 0.5, 1, 1.5, 2, 2.5, 3, 3.5, 4, 4.5, 4.8, 4.9, 5, 5.5, 6, 6.5, 7, 7.5, 8, 8.5, 9, 9.5 and 10 mg pipamperone per day is used in the treatment of mood disorders, such as depression.

In a further preferred embodiment, dosages of more than about 10 mg to about 20 mg per day are used in the treatment of mood disorders, such as depression, preferably in treating adults. Preferably, about 10, 11, 12, 12.5, 13, 14, 15, 16, 17, 17.5, 18, 19, 19.5 and 20 mg pipamperone per day is used in the treatment of mood disorders, such as depression.

Dosages between about 0.00125 mg and about 0.25 mg pipamperone per kg bodyweight per day are used in the treatment of mood disorders, such as depression, such as, for instance, 0.00125, 0.0025, 0.005, 0.01, 0.015, 0.02, 0.025, 0.03, 0.035, 0.04, 0.045, 0.05, 0.055, 0.06, 0.0625, 0.10, 0.125, 0.15, 0.16, 0.17, 0.175, 0.18, 0.19, 0.195, 0.20, 0.22, 0.23, 0.24 and 0.25 mg pipamperone per day per kg bodyweight is used.

It will be appreciated that the daily doses may be unitary doses.

Accordingly, the present invention relates to the use of pipamperone or a pharmaceutically acceptable salt thereof for the preparation of a medicament for treating a mood disorder, wherein said pipamperone or a pharmaceutically acceptable salt is to be administered to a patient in a daily dose ranging between 0.1 and about 20 mg of the active ingredient.

Accordingly, the present invention relates to a pharmaceutical composition comprising pipamperone for use in treating mood disorders, wherein said pipamperone is to be administered to a patient in a daily dose ranging between about 0.1 and about 20 mg of the active ingredient.

Accordingly, the present invention relates to pipamperone or a pharmaceutically acceptable salt thereof for use in treating mood disorders, wherein said pipamperone or pharmaceutically acceptable salt is to be administered to a patient in a daily dose ranging between about 0.1 and about 20 mg of the active ingredient.

Accordingly, the present invention relates to a pharmaceutical composition comprising pipamperone, or a pharmaceutically acceptable salt thereof, at between 0.1 mg and about 20 mg of the active ingredient. Preferably said pipamperone, or a pharmaceutically acceptable salt thereof, is provided in a unitary dose of between 0.1 and about 20 mg of the active ingredient. More preferably, said pharmaceutical composition is characterised in that it is a unit dose preparation containing 0.1 to about 20 mg pipamperone, more preferably, a unit dose preparation containing not more than 15 mg pipamperone, more preferably, a unit dose preparation containing not more than 12.5 mg pipamperone, preferably 10 mg.

According to a further embodiment, the invention relates to the use of pipamperone for the preparation of a medicament for treating mood disorders, and in particular depression, whereby pipamperone is administered at a dose of more than 0.1 to about 20 mg per day in the treatment of mood disorders, and in particular depression. Preferably, pipamperone is administered for at least 1, 2, 3, 4, 5, 6, 7, 8, 9, 10, 11, 12, 13 or 14 days, even more preferably, pipamperone is administered for at least 2, 3, 4 or 5 weeks, or even 1, 2, 3, 4, 5, 6, 7 8, 9, 10, 11 or 12 months or even longer, according to the patient's need.

Pipamperone is preferably administered once a day. In a further embodiment, pipamperone is administered 2, or 3 or even 4 times a day. It will be understood that, apart from daily doses, the compounds can be administered by other schedules. For instance, the present invention also contemplates depot injection, in which a long acting form of the active compound is injected into the body, such as the muscles. From there the active compound slowly enters the rest of the body, so one injection can last from 1 to 4 weeks or even multiple months. Other form of dosage administrations relate to “once-a-week” pills, in which the ingredient is slowly released over a period of a week, and slow-release patches, e.g. a CDS (Continuous Delivery System), or Once-a-Day Transdermal Patches.

It will be understood that the total dose of pipamperone per day of any dosage regime is as described above, e.g. from about 0.1 mg per day to about 20 mg pipamperone per day.

The terms “treatment”, “treating”, and the like, as used herein include amelioration or elimination of a developed mental disease or condition, in particular depression, once it has been established or alleviation of the characteristic symptoms of such disease or condition. As used herein these terms also encompass, depending on the condition of the patient, preventing the onset of a mood disorder or of symptoms associated with a mood disorder, including reducing the severity of a mood disorder or symptoms associated therewith prior to affliction with said mood disorder. Such prevention or reduction prior to affliction refers to administration of the compound or composition of the invention to a patient that is not at the time of administration afflicted with a mood disorder, such as depression. “Preventing” also encompasses preventing the recurrence or relapse-prevention of depression or of symptoms associated therewith, for instance after a period of improvement. It should be clear that mental conditions of a mood disorder may be responsible for physical complaints. In this respect, the term “treating” also includes prevention of a physical disease or condition or amelioration or elimination of the developed physical disease or condition once it has been established or alleviation of the characteristic symptoms of such conditions.

As used herein, the term “medicament” also encompasses the terms “drug”, “therapeutic”, “potion” or other terms which are used in the field of medicine to indicate a preparation with therapeutic or prophylactic effect.

Patients receiving antidepressant monotherapy may be or become partially or totally resistant to treatment in 10 to 30 percent of cases. “Pooping-out” can occur with any conventional antidepressant. Relapse or recurrence of depression while still taking medication (i.e., breakthrough) can also occur. While there is no definitive answer as to why this happens, it may be a case of the patient developing tolerance to the drug. The most commonly strategies used to deal with this problem include augmentation with a second drug, raising the dose or switching to another drug entirely.

A change from one antidepressant to another in the same class has not produced impressive response rates among depressed patients. Although some studies suggest that switching to an antidepressant with a different mechanism of action is often associated with a better response rate, however, the results thereof are not convincing. If drug-switching is chosen as the method of therapy, patients should be closely monitored for possible drug interactions or other adverse effects. This is particularly true if the half-life of the first agent is quite long (e.g., fluoxetine [Prozac]) and another SSRI is begun before a prudent “wash-out” period has occurred. This situation may sometimes result in serotonin syndrome (toxic levels of central nervous system serotonin that result in hyper-alertness, agitation, confusion, restlessness, myoclonus, hyperreflexia, diaphoresis, shivering, tremor and, possibly, death.)

The present inventors found that pipamperone which binds to the 5-HT2A receptor with a pKi of at least 8 but for which the binding affinity, i.e. pKi, towards other 5HT receptors is less than 8 in combination with a high selective affinity for the D4 receptor, i.e. which bind to the D4 receptor with a pKi of at least 8 but for which the binding affinity, i.e. pKi, towards other dopamine receptors is less than 8 also show an improved effect on treatment refractory patients with mood disorders. Because of high specific binding affinity, pipamperone can be used at low doses. At these low doses, adverse effects are minimized and/or precluded (see e.g. WO2005/053796, which is specifically incorporated by reference in its entirety).

The term “other 5HT receptors” as used herein relate to, for instance, 5-HT1 receptors (e.g. 5-HT1A, 5-HT1 B, 5-HT1D, 5-HT1 E, 5-HT1F), 5-HT2B, 5-HT2C, 5-HT6 (rat) and 5-HT7 (rat). The expression “selective affinity for the 5-HT2A receptor” relates to a receptor having a higher affinity for the 5-HT2A receptor than for other 5-HT receptors. The expression “selective affinity for the D4 receptor” means that the receptor has a higher affinity for the dopamine D4 receptor than for other dopamine receptors. The term “other dopamine receptors” relates to, for instance, D1, D2 and D3 dopamine receptors. pKi values of test compounds for dopamine receptors as well as 5-HT2A receptors can be measured using commonly known assays.

Table 1 illustrates the selective affinity of for instance pipamperone for the 5-HT2A and for the D4 receptor. In addition, Table 1 also illustrates the low or absence of affinity of pipamperone for other receptors such as the adrenergic receptors Alpha 1A, Alpha 2A, Alpha 2B, Alpha 2C, Beta 1, Beta 2, and the histamine receptor H1. As such, treating patients with pipamperone will provide for less side-effects which otherwise result from simultaneous stimulation of other receptors.

The low dosage which can be used in pipamperone treatment, as already described earlier, contributes to the high selective affinity of the compound towards the 5-HT2A receptor and the D4 receptor and therefore also to the efficacy of the treatment.

Accordingly, the present invention relates to the use as described herein, characterised in that the pharmaceutical composition is for treatment of patients diagnosed with a mood disorder who have failed to respond to initial treatment with an antidepressant, such as an SSRI, SNDRI or SNRT. More in particular, the present invention relates to the use as described above, characterised in that the pharmaceutical composition is for treatment of patients with a mood disorder, such as major depression disorder who have failed to respond to initial treatment with an antidepressant, such as an SSRI, SNDRI or SNRI. Accordingly, the present invention relates to the use as described herein, characterised in that the pharmaceutical composition is for treatment of treatment refractory patients to an SSRI, SNDRI or SNRI. More in particular, the present invention relates to the use as described above, characterised in that the pharmaceutical composition is for treatment of major depression in treatment refractory patients to an SSRI, SNDRI or SNRI.

The term “treatment refractory” patient is used as common in the art, such as, for instance, as understood by the clinician or physician monitoring and/or treating a patient. This term includes patients who have failed to respond to initial treatment, patients who are partially or totally resistant to treatment, patients with recurrent mood disorder, and patients pooping-out. Patients who failed to respond to initial treatment include patients not ameliorating after for instance 2 weeks of treatment, as diagnosed by criteria described above.

The compositions according to the invention may be pharmaceutical compositions or formulations. The pharmaceutical compositions or formulations may be packed together, such as, for instance, in a box, on a strip, e.g. a blister strip, etc. The present invention relates to pharmaceutical composition comprising pipamperone, and the use thereof, as described herein, characterised in that said pharmaceutical composition is an oral formulation, preferably a tablet.

Preferably, the composition is a pill, powder or solution comprising pipamperone at the doses of the invention. This will ease in administration and management.

In this invention, the term “antagonist” refers to an interaction between chemicals in which one partially or completely inhibits the effect of the other, in particular agents having high affinity for a given receptor, but which do not activate this receptor. In this invention, the term “inverse agonist” refers to a ligand which produces an effect opposite to that of the agonist by occupying the same receptor. In this invention, the term “agonist” relates to an agent which both binds to a receptor and has an intrinsic effect. In this invention, the term “partial agonist” relates to an agent with lower intrinsic activity than a full agonist, and which produces a lower maximum effect.

The term “active metabolite” as used herein relates to a therapeutically active compound produced by the metabolism of a parent drug. Drugs administered to treat diseases are usually transformed (metabolized) within the body into a variety of related chemical forms (metabolites), some of which may have therapeutic activity (an active metabolite).

The compounds according to the invention may be chemical or biological in nature, or may be chemically synthesised.

The present invention also encompasses the use of pipamperone administered in the form of a pharmaceutically acceptable salt in admixture with a suitable pharmaceutically acceptable excipient.

To prepare the pharmaceutical compositions, comprising pipamperone an effective amount of the active ingredients, in acid or base addition salt form or base form, is combined in admixture with a pharmaceutically acceptable carrier, which can take a wide variety of forms depending on the form of preparation desired for administration. These pharmaceutical compositions are desirably in unitary dosage form suitable, for administration orally, nasal, rectally, percutaneously, transdermally, by parenteral, intramuscular, intravascular injection or intrathecal administration. For example, in preparing the compositions in oral dosage form, any of the usual pharmaceutical media may be employed, such as, for example, water, glycols, oils, alcohols and the like in the case of oral liquid preparations such as suspensions, syrups, elixirs and solutions; or solid carriers such as starches, sugars, kaolin, lubricants, binders, disintegrating agents and the like in the case of powders, pills, capsules and tablets. Because of their ease in administration, tablets and capsules represent the most advantageous oral dosage unit form, in which case solid pharmaceutical carriers are obviously employed. For parenteral compositions, the carrier will usually comprise sterile water, at least in large part, though other ingredients, for example, to aid solubility, may be included.

Accordingly, the present invention relates to the use of pipamperone comprising between 0.1 mg and about 20 mg for the preparation of a pharmaceutical composition.

The pharmaceutical compounds for treatment are intended for parenteral, topical, oral or local administration and generally comprise a pharmaceutically acceptable carrier and an amount of the active ingredient sufficient to reverse or prevent the bad effects of mental disorders. The carrier may be any of those conventionally used and is limited only by chemico-physical considerations, such as solubility and lack of reactivity with the compound, and by the route of administration.

Preferably, the compounds of the invention are provided as a pharmaceutically acceptable salt. Examples of pharmaceutically acceptable acid addition salts for use in the present inventive pharmaceutical composition include those derived from mineral acids, such as hydrochloric, hydrobromic, phosphoric, metaphosphoric, nitric and sulfuric acids, and organic acids, such as tartaric, acetic, citric, malic, lactic, fumaric, benzoic, glycolic, gluconic, succinic, p-toluenesulphonic acids, and arylsulphonic, for example. The present invention relates specifically to pipamperone and the use thereof, characterised in that said pipamperone is pipamperone dihydrochloride or pipamperone acetate.

The pharmaceutically acceptable excipients described herein, for example, vehicles, adjuvants, carriers or diluents, are well-known to those who are skilled in the art and are readily available to the public. It is preferred that the pharmaceutically acceptable carrier be one that is chemically inert to the active compounds and one that has no detrimental side effects or toxicity under the conditions of use.

The following formulations for oral, aerosol, parenteral, subcutaneous, intravenous, intramuscular, interperitoneal, rectal, and vaginal administration are merely exemplary and are in no way limiting. Overall, the requirements for effective pharmaceutical carriers for parenteral compositions are well known to those of ordinary skill in the art. See Pharmaceutics and Pharmacy Practice, J. B. Lippincott Company, Philadelphia, Pa., Banker and Chalmers, eds., pages 238-250, (1982), and ASHP Handbook on Injectable Drugs, Toissel, 4th ed., pages 622-630 (1986). Topical formulations, including those that are useful for transdermal drug release, are well-known to those of skill in the art and are suitable in the context of the present invention for application to skin.

Formulations, comprising pipamperone suitable for oral administration require extra considerations considering the nature of the compounds and the possible breakdown thereof if such compounds are administered orally without protecting them from the digestive secretions of the gastrointestinal tract. Such a formulation can consist of (a) liquid solutions, such as an effective amount of the compound dissolved in diluents, such as water, saline, or orange juice; (b) capsules, sachets, tablets, lozenges, and troches, each containing a predetermined amount of the active ingredient, as solids or granules; (c) powders; (d) suspensions in an appropriate liquid; and (e) suitable emulsions. Liquid formulations may include diluents, such as water and alcohols, for example, ethanol, benzyl alcohol, and the polyethylene alcohols, either with or without the addition of a pharmaceutically acceptable surfactant, suspending agent, or emulsifying agent. Capsule forms can be of the ordinary hard- or soft-shelled gelatine type containing, for example, surfactants, lubricants, and inert fillers, such as lactose, sucrose, calcium phosphate, and corn starch. Tablet forms can include one or more of lactose, sucrose, mannitol, corn starch, potato starch, alginic acid, microcrystalline cellulose, acacia, gelatine, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavouring agents, and pharmacologically compatible excipients. Lozenge forms can comprise the active ingredient in a flavour, usually sucrose and acacia or tragacanth, as well as pastilles comprising the active ingredient in an inert base, such as gelatine and glycerine, or sucrose and acacia, emulsions, gels, and the like containing, in addition to the active ingredient, such excipients as are known in the art.

Accordingly, the present invention relates to tablets comprising between 0.1 and about 20 mg pipamperone or a pharmaceutically acceptable salt thereof, and optionally lactose, corn starch, saccharose, talc, and/or magnesium-stearate.

The compounds of the present invention, alone or in combination with other suitable components, can be made into aerosol formulations to be administered via inhalation. For aerosol administration, the compounds are preferably supplied in finely divided form along with a surfactant and propellant. Typical percentages of compounds are 0.01%-20% by weight, preferably 1%-10%. The surfactant must, of course, be nontoxic, and preferably soluble in the propellant. Representative of such agents are the esters or partial esters of fatty acids containing from 6 to 22 carbon atoms, such as caproic, octanoic, lauric, palmitic, stearic, linoleic, linolenic, olesteric and oleic acids with an aliphatic polyhydric alcohol or its cyclic anhydride. Mixed esters, such as mixed or natural glycerides may be employed. The surfactant may constitute 0.1%-20% by weight of the compounds, preferably 0.25-5%. The balance of the compounds is ordinarily propellant. A carrier can also be included as desired, e.g., lecithin for intranasal delivery. These aerosol formulations can be placed into acceptable pressurized propellants, such as dichlorodifluoromethane, propane, nitrogen, and the like. They also may be formulated as pharmaceuticals for non-pressured preparations, such as in a nebulizer or an atomizer. Such spray formulations may be used to spray mucosa.

It should be clear that the compounds and compositions described herein are useful for treating any patient in need thereof, in particular humans.

Mood disorders, in particular depression, can be treated using compounds having a high selective affinity for the 5-HT2A and D4 receptor, for instance pipamperone, The present invention thus relates to the use of pipamperone or a pharmaceutically acceptable salt thereof for the preparation of a medicament for treating mood disorders, including depression, characterized in that pipamperone or said pharmaceutically acceptable salt thereof is administered to a patient in a daily dose ranging between 0.1 and about 20 mg of the active ingredient.

The present invention also relates to the use of pipamperone for the treatment of major depression disorder characterised in that it is used in a daily dose of less than 20 mg of pipamperone, preferably in that it is used in a daily dose of 15 mg or less of pipamperone. The present invention also relates to the use for the treatment of major depression disorder characterised in that it is used in a daily dose of 10 mg of pipamperone.

Finally, the present invention also relates to a method for treating mood disorder comprising administering pipamperone between 0.1 and less than 20 mg per day.

The disclosure of all patents, publications (including published patent publications), and database accession numbers and depository accession numbers referenced in this specification are specifically incorporated herein by reference in their entirety to the same extent as if each such individual patent, publication, and database accession number, and depository accession number were specifically and individually indicated to be incorporated by reference.

The mental disorders which can be treated using pipamperone are chosen from mood disorders.

The invention, now being generally described, will be more readily understood by reference to the following tables and examples, which are included merely for purposes of illustration of certain aspects and embodiments of the present invention and are not intended to limit the invention.

Short Description of the Tables

Table 1: In Table 1, the pKi values of test compounds are given for each of the dopamine receptors, 5HT receptors, adrenergic receptors and the histamine1 receptor.

Table 2: Set-up of a clinical trial comprising for treatment groups.

### EXAMPLES

### Example 1

### Pipamperone Treatment in Major Depressive Disorder: a Placebo and Active Controlled Clinical Trial

Table 2 represents the set-up of a clinical trial comprising for treatment groups:

Group Cit—Active/Day 0 represents the group receiving 20 mg citalopram, twice a day, starting the first day (Day 0) of active treatment in the clinical trial in a forced titration regime. This administration regime is also indicated as conventional mono therapy.

Group Pip—Active/Day 0 represents the group receiving 5 mg pipamperone, twice a day, starting the first day (Day 0) of active treatment in the clinical trial.

Group Plc—Non-active/Day 0 represents the group receiving a placebo, twice a day, starting the first day (Day 0) of active treatment in the clinical trial.

All subjects also undergo a placebo (PLC) run-in therapy, administered during a period of about 7 days before the active treatment starts.

During daily (D), weekly (W) or monthly (M) visits, several parameters are measured.

Under NECT is to be understood: Neuronal E-clinical Trial =Vesalius Expert development for this trial which includes the bottom-up measurement of:
In- and exclusion-criteria
Functional status evaluation
Medical history
(Pre-)treatment signs & symptoms
DSM-IV rules for diagnosis & efficacy
HDRS-28 (Hamilton Depression Rating Scale—28 items)
Medical resource utilisation
Pre-trial & Concomitant medication
Drug administration
(Serious) Adverse events
Admission to the acute and extension phase of treatment
Right flow of the trial

### Example 2

### Pipamperone: Therapeutic Use in Treatment Refractory Patients With Major Depression

Purpose Pipamperone, administered to patients in a dose ranging between 1 and 20 mg is claimed via its specific pharmacological properties to have an antidepressant effect. The mechanism of pipamperone has to deal with (i) the selective affinity for the dopamine-4 (D4) receptor with a pKi value equal to or higher than 8 towards the D4 receptor and less than 8 towards other dopamine receptors, and (ii) the selective affinity for the 5-HT2A receptor with a pKi value equal to or higher than 8 towards the 5-HT2A receptor and less than 8 towards other 5HT receptors. This semi-naturalistic open label study investigated the efficacy and tolerability of low dose pipamperone in the treatment of patients with treatment refractory major depression.

Details

Design: Semi-naturalistic i.e. inclusion of every ‘natural’ patient in an outpatient practice but without concomitant use of mood enhancing drugs, open label

Control: No

Phase: Phase Ila—preliminary Proof of Concept

Location: Belgium —Research Centre ANIMA, Aiken

End Points: Assessment scale scores, Hamilton Depression Rating Scale 17 items, Reduction, Response, Remission

Medication: Exclusion of mood stabilisers, antipsychotics (typical and atypical) and other antidepressants

Subjects Patients have a major depressive disorder according to DSM-IV criteria, with or without a chronic course and a treatment refractory state towards SSRIs.

Treatments

PIP—pipamperone from DAY 0

| Drug/Treatment | Dose (total) | Route | Duration |
|---|---|---|---|
| Pipamperone¹ | Pip.: 1-20 mg/day | PO | 8 weeks |

¹Pipamperone (Pip) dosage was adjusted according to clinical response.

1. Pipamperone (Pip) dosage was adjusted according to clinical response.

Results

The results for the PIP treatment are compared with:

(1) standard efficacy of antidepressants in clinical trials according to Khan et al. (2000), in “Symptom Reduction and Suicide Risk in Patients Treated With Placebo in Antidepressant Clinical Trials” (Arch. of General Psychiatry, Vol. 57, April 2000).

(2) HDRS-17 change from baseline vs SNRI (duloxetine) in Major Depression; the SNRI (duloxetine) treatment was 40-120 mg/day (n=152) according to Goldstein et al., (Clin. Psychiatry).

(3) remission rates (HDRS-17 <=7) vs SSRIs vs placebo in Major Depression; treatment with SSRIs is according to a meta-analysis of Thase et al. (Br. J. Psychiatry (2001) 178:234-241). Treatment with placebo is according to a meta-analysis of Thase et al. (Br. J. Psychiatry (2001) 178:234-241).

(4) WO2005/053796

Adverse Events

Adverse events are assessed on: body as a whole, central and peripheral nervous system, gastrointestinal, musculoskeletal, psychiatric, respiratory, skin and appendages, vascular and urinary, taking into account discontinued treatment due to adverse events. Laboratory parameters, ECG, bodyweight and vital signs are not measured since this is a naturalistic study.

Study Messages

The anti-depressant effect of pipamperone at an extremely unconventional low dose is apparent.

The anti-depressant effect on treatment refractory patients of pipamperone is apparent.

The pipamperone is generally well tolerated in patients with depression, i.e. at least no specific adverse events are expected by pipamperone at the doses used in the study.

Sedative effects of pipamperone, which are observed at higher doses, are lacking.

### Example 3

### POC Process for Major Depressive Disorder

Concept: the high selective 5-HT2A/D4 antagonist pipamperone in treating major depressive disorder at a dose of pipamperone between 1-20 mg/day

Objectives: Demonstrating that this therapy has:
the potency of being a treatment standard for depression by having a clinical significant reduction of the total score of the Hamilton Depression Rating Scale—17 items (HDRS-17) after 8 weeks of therapy. This stands for an added medium demission of 2.5 points on the total score of the HDRS-17 compared to placebo; —a more sustained therapeutic effect than the conventional therapy by preventing significant more relapses during 48 weeks following the acute treatment; and/or
a complete neutral safety profile, e.g. there are no more clinical relevant adverse events in the therapy than in admission of placebo.

Process: the following different steps are implemented to reach out for these objectives (see also Table 2):

(1) an naturalistic open label study (n=>20) on a depressive population with a normal variability of medical and psychiatric history, course of depression, earlier and concomitant therapy admitting the golden standard antidepressant citalopram 20-40 mg/day versus a dose of 1-20 mg/day of pipamperone.

(2) a 16 weeks placebo controlled randomised three armed study of each 36 patients with a major depressive disorder admitting: from day 0: placebo or pipamperone (PIP) 5 mg/day or an active antidepressant compound;

By including rigorous control groups (placebo and active comparator; see Table 2) this clinical trial is evaluated as a proof of concept of the antidepressant effect of PIP treatment, since the inclusion/exclusion of:
a negative trial, i.e. no significant difference between the placebo and active treatment with the comparator;
a failed trial, i.e. inferiority of the studied treatment i.e. the PIP, compared to the active treatment with the comparator.

(3) an active controlled randomised relapse prevention study following the POC trial during another 36 weeks with three arms, which is formed by:
continuation of the active conventional mono therapy;
randomising the patients with a PIP therapy in a group with an active PIP therapy and with a placebo treatment.

TABLE 1

| | D1 | D2 | D3 | D4 | 5HT_{1A} | 5HT_{1B} | 5HT_{1D} | 5HT_{1E} | 5HT_{1F} | 5HT_{2A} | 5HT_{2B} |
|---|---|---|---|---|---|---|---|---|---|---|---|
| ORG5222 | 8-9 | 8-9 | 8-9 | 8-9 | 8-9 | 8-9 | 8-9 | 7-8 | 0 | >9 | >9 |
| Zotepine | 0 | 8-9 | 8-9 | 7-8 | 6-7 | 7-8 | 7-8 | 6-7 | 0 | 8-9 | 0 |
| Fluparoxan | 0 | <6 | <6 | 0 | 6-7 | <6 | <6 | 0 | 0 | <6 | 0 |
| Olanzapine | 7-8 | 7-8 | 7-8 | 7-8 | <6 | 6-7 | 6-7 | <6 | 6-7 | 8-9 | 8-9 |
| Clozapine | 7-8 | 6-7 | 6-7 | 7-8 | 6-7 | 6-7 | 6-7 | 6-7 | 6-7 | 8-9 | 8-9 |
| S16924 | 0 | 7-8 | 7-8 | 7-8 | 8-9 | 0 | 0 | 0 | 0 | >9 | 8-9 |
| S18327 | 7-8 | 7-8 | 6-7 | 8-9 | 7-8 | 0 | 0 | 0 | 0 | 8-9 | 0 |
| Amperozide | 6-7 | 6-7 | 6-7 | <6 | <6 | 0 | 0 | 0 | 0 | 8-9 | 0 |
| GGR218231 | <6 | 7-8 | >9 | <6 | 6-7 | <6 | <6 | 0 | 0 | <6 | <6 |
| Sertindole | 7-8 | 8-9 | 8-9 | 7-8 | 6-7 | 7-8 | 7-8 | 6-7 | 6-7 | >9 | 0 |
| MDL100.907 | 6-7 | <6 | <6 | 6-7 | <6 | 0 | 0 | 0 | 0 | >9 | 0 |
| Haloperidol | 8-9 | >9 | 8-9 | 8-9 | <6 | 6-7 | <6 | <6 | <6 | 6-7 | <6 |
| Tiospirone | 7-8 | 8-9 | 8-9 | 8-9 | 8-9 | 0 | 0 | 0 | 0 | >9 | 0 |
| Raciopride | <6 | 8-9 | 8-9 | <6 | <6 | 0 | 0 | 0 | 0 | 6-7 | 0 |
| Fluspirilene | 0 | >9 | 8-9 | 8-9 | 7-8 | <6 | <6 | <6 | 0 | <6 | 0 |
| Ocaperidone | 7-8 | >9 | 8-9 | 8-9 | 7-8 | 0 | 0 | 0 | 0 | >9 | 0 |
| Risperidone | 7-8 | 8-9 | 7-8 | 8-9 | 6-7 | 8-9 | 6-7 | <6 | <6 | >9 | 0 |
| S33084 | 6-7 | 7-8 | >9 | <6 | <6 | 6-7 | 6-7 | 0 | 0 | 6-7 | 6-7 |
| L741626 | 6-7 | 8-9 | 7-8 | 6-7 | <6 | <6 | <6 | 0 | 0 | 6-7 | 6-7 |
| Seroquel | 6-7 | 6-7 | 6-7 | <6 | 6-7 | <6 | <6 | <6 | <6 | 6-7 | 6-7 |
| Yohimbine | 0 | 6-7 | <6 | 0 | 7-8 | 6-7 | 7-8 | 0 | 0 | <6 | 0 |
| Ziprasidone | 8-9 | 8-9 | 7-8 | 7-8 | 8-9 | >9 | 8-9 | 6-7 | 0 | >9 | 8-9 |
| Pipamperone | 0 | 6-7 | 6-7 | 8-9 | <6 | 6-7 | 6-7 | <6 | <6 | 8-9 | 0 |

| | 5HT_{2C} | 5HT₆rat | 5HT₇rat | Alpha1A | Alpha2A | Alpha2B | Alpha2C | Beta1 | Beta2 | H1 |
|---|---|---|---|---|---|---|---|---|---|---|
| ORG5222 | >9 | >9 | >9 | >9 | 8-9 | >9 | 7-8 | <6 | <6 | >9 |
| Zotepine | 0 | 0 | 0 | 0 | 6-7 | 8-9 | 6-7 | <6 | <6 | >9 |
| Fluparoxan | <6 | 0 | 0 | 6-7 | 8-9 | 8-9 | 8-9 | 0 | 0 | 0 |
| Olanzapine | 8-9 | 7-8 | 6-7 | 7-8 | 6-7 | 6-7 | 6-7 | <6 | <6 | >9 |
| Clozapine | 7-8 | 7-8 | 7-8 | 8-9 | 7-8 | 7-8 | 7-8 | <6 | <6 | >9 |
| S16924 | 7-8 | 7-8 | 7-8 | 8-9 | 6-7 | 7-8 | 6-7 | <6 | <6 | 0 |
| S18327 | 6-7 | 0 | 0 | >9 | 6-7 | 0 | 0 | 0 | 0 | 0 |
| Amperozide | <6 | 0 | 0 | 7-8 | <6 | 0 | 0 | 0 | 0 | 0 |
| GGR218231 | <6 | 0 | 0 | <6 | <6 | 0 | 0 | 0 | 0 | 0 |
| Sertindole | 8-9 | 0 | 0 | >9 | 6-7 | 6-7 | 6-7 | <6 | <6 | 6-7 |
| MDL100.907 | 7-8 | 0 | 0 | <6 | <6 | 0 | 0 | 0 | 0 | 0 |
| Haloperidol | <6 | <6 | 6-7 | 8-9 | <6 | 6-7 | <6 | <6 | <6 | 6-7 |
| Tiospirone | 8-9 | 0 | 0 | >9 | 6-7 | 0 | 0 | 0 | 0 | 0 |
| Raciopride | <6 | 0 | 0 | <6 | <6 | 0 | 0 | 0 | 0 | 0 |
| Fluspirilene | 0 | 0 | 0 | 0 | 6-7 | 7-8 | 7-8 | 6-7 | 6-7 | 7-8 |
| Ocaperidone | 7-8 | 0 | 0 | >9 | 0 | 0 | 0 | 0 | 0 | 0 |
| Risperidone | 7-8 | 0 | 0 | >9 | 7-8 | 8-9 | 8-9 | <6 | <6 | 7-8 |
| S33084 | 7-8 | 0 | 0 | 6-7 | <6 | 0 | 0 | 0 | 0 | 0 |
| L741626 | <6 | 0 | 0 | 6-7 | <6 | 0 | 0 | 0 | 0 | 0 |
| Seroquel | 6-7 | 0 | 6-7 | 7-8 | <6 | 7-8 | 6-7 | <6 | <6 | 8-9 |
| Yohimbine | <6 | 0 | 0 | 6-7 | 8-9 | 8-9 | >9 | <6 | <6 | 0 |
| Ziprasidone | 8-9 | 7-8 | 8-9 | 8-9 | 6-7 | 7-8 | 7-8 | <6 | <6 | 7-8 |
| Pipamperone | 0 | 0 | 0 | 0 | 6-7 | 7-8 | 6-7 | <6 | <6 | <6 |

TABLE 2

| | ACUTE PHASE** | | | | | | EXTENSION PHASE*** | | | | | | | FOLLOW-UP PHASE | | | | | |
|---|---|---|---|---|---|---|---|---|---|---|---|---|---|---|---|---|---|---|---|
| | VISITS | | | | | | | | | | | | | | | | | | |
| | V1 | V2 | | | | | | | | | | | | | | | | | |
| | Screen | Base- | | | | | | | | | | | | | | | | | |
| | minus | line | V3 | V4 | V5 | V6 | V7 | V8 | V9 | V10 | V11 | V12 | V13 | V14 | V15 | V16 | V17 | V18 | V19 |
| Day/Week/Month | D7 | D0 | D4 | D7 | W2 | W3 | W4 | W6 | W8 | W10 | W12 | W16 | W20 | W24 | M8 | M10 | M12 | W1 | W2 |
| TREATMENT GROUP | | | | | | | | | | | | | | | | | | | |
| Group Plc-Non active/D0 | A | A | | | | | | | | | | | | | | | | A | A |
| Group Pip-Active/D0 | A | B | | | | | | | | | | | | | | | | A | A |
| Group Cit-Active/D0 | A | C | | | | | | | | | | | | | | | | A | A |
| Informed Consent | x | | | | | | | | | | | | | | | | | | |
| NECT* | x | x | x | x | x | x | x | x | x | x | x | x | x | x | x | x | x | x | x |
| Vital Signs/Weight | x | | | | | | | | | | | | | x | | | x | | x |
| LAB | x | | | | | | | | | | | | | x | | | x | | x |
| ECG | x | | | | | | | | | | | | | x | | | x | | x |
| Phys Exam | x | | | | | | | | | | | | | | | | | | |
| Alc/Drugs Screen | x | | | | | | | | | | | | | x | | | x | | x |
| CGI-S**** | x | x | x | x | x | x | x | x | x | x | x | x | x | x | x | x | x | x | x |
| Q-LES-Q***** | | | | | | | | | | | | | | | | | | | |

Treatment regimen:
A: PLC + PLC
B: 2 × (PLC + PIP(5 mg))/d
C: 2 × (CIT(20 mg) + PLC))/d
*Neuronal E-Clinical Trial = Vesalius Expert Development for this Trial which includes the bottom-up measurement of:
**Entering Acute Phase: only NON-placebo responders as defined by the DSM-IV criteria of efficacy
***Entering Extension Phase: only remittors as defined by the DSM-IV criteria of efficacy
****CGI-S: Clinical Global Impressions-Improvement Scale
*****Q-LES-Q: Quality of Life, Enjoyment and Satisfaction Questionnaire

1. A method for treating a mood disorder in a patient comprising administering pipamperone or a pharmaceutically acceptable salt thereof to the patient in a daily dose ranging between 0.1 and about 20 mg.

2. A pharmaceutical composition comprising pipamperone for use in treating a mood disorder, wherein pipamperone is to be administered to a patient in a daily dose ranging between 0.1 and about 20 mg.

3. A pharmaceutical composition comprising between 0.1 mg and about 20 mg pipamperone, or a pharmaceutically acceptable salt thereof.

4. The pharmaceutical composition according to claim 3, wherein the composition is formulated in a unit dose preparation containing 0.1 to about 20 mg pipamperone.

5. The pharmaceutical composition according to claim 3, wherein the composition is an oral formulation.

6. A tablet comprising between 0.1 and about 20 mg pipamperone or a pharmaceutically acceptable salt thereof, and optionally lactose, corn starch, saccharose, talc, and/or magnesium-stearate.

7. A method of preparing a pharmaceutical composition comprising formulating between 0.1 and about 20 mg pipamperone in a pharmaceutical composition.

8. The method according to claim 7. wherein pipamperone is used as pipamperone dihydrochloride or pipamperone acetate.

9. The method according to claim 7, wherein the pharmaceutical composition is for treatment of a mood disorder.

10. The method according to claim 1, wherein the patient is refractory to a selective serotonin reuptake inhibitor (SSRI), a serotonin, norepinephrine and dopamine reuptake inhibitor (SNDRI) or a serotonin and norepinephrine reuptake inhibitor (SNRI).

11. The method according to claim 1, wherein pipamperone is administered in a daily dose of less than 20 mg of pipamperone.

12. The method according to claim 1, wherein pipamperone is administered in a dose between about 0.1 and less than 20 mg per day.

13. The pharmaceutical composition according to claim 5, wherein the oral formulation is a tablet.

14. The tablet according to claim 6 comprising between 0.1 and about 20 mg pipamperone or a pharmaceutically acceptable salt thereof, and lactose, corn starch, saccharose, talc, and/or magnesium-stearate.

15. The pharmaceutical composition according to claim 2, wherein the pharmaceutical composition is for treatment of a patient who is refractory to a selective serotonin reuptake inhibitor (SSRI), a serotonin, norepinephrine and dopamine reuptake inhibitor (SNDRI) or a serotonin and norepinephrine reuptake inhibitor (SNRI).

16. The pharmaceutical composition according to claim 3, wherein the pharmaceutical composition is for treatment of a patient who is refractory to a selective serotonin reuptake inhibitor (SSRI), a serotonin, norepinephrine and dopamine reuptake inhibitor (SNDRI) or a serotonin and norepinephrine reuptake inhibitor (SNRI).

17. The tablet according to claim 6, for treatment of a patient who is refractory to a selective serotonin reuptake inhibitor (SSRI), a serotonin, norepinephrine and dopamine reuptake inhibitor (SNDRI) or a serotonin and norepinephrine reuptake inhibitor (SNRI).

18. The method according to claim 7, wherein the pharmaceutical composition is for treatment of a patient who is refractory to a selective serotonin reuptake inhibitor (SSRI), a serotonin, norepinephrine and dopamine reuptake inhibitor (SNDRI) or a serotonin and norepinephrine reuptake inhibitor (SNRI).
